(19) United States
(12) Patent Application Publication
(10) Pub. No.: US 2025/0255781 A1
(43) Pub. Date: Aug. 14, 2025

(54) PROCESSES FOR PREPARATION OF 3-D PRINTED PHARMACEUTICAL PRODUCTS

(71) Applicant: BRISTOL-MYERS SQUIBB COMPANY, PRINCETON, NJ (US)

(72) Inventors: ATABAK TABRIZ, LONDON (GB); DENNIS DOUROUMIS, LONDON (GB); HO-WAH HUI, BASKING RIDGE, NJ (US); SUMIT KUMAR, YARDLEY, PA (US); YUCHUAN GONG, VERNON HILLS, IL (US)

(73) Assignee: BRISTOL-MYERS SQUIBB COMPANY, PRINCETON, NJ (US)

(21) Appl. No.: 18/857,946

(22) PCT Filed: Apr. 18, 2023
(86) PCT No.: PCT/US2023/065869

§ 371 Date: Oct. 18, 2024

Related U.S. Application Data
(60) Provisional application No. 63/332,397, filed on Apr. 19, 2022.

(51) Int. Cl. A61J 3/00; A61K 9/20; A61K 31/192; B33Y 10/00; B33Y 80/00

(52) U.S. Cl. CPC A61J 3/005; A61K 9/2095; A61K 31/192; B33Y 10/00; B33Y 80/00

(57) ABSTRACT

This invention relates to methods of producing 3-D printed pharmaceutical products.

### CROSS REFERENCE TO RELATED APPLICATIONS

This application is entitled to priority pursuant to 35 U.S.C. § 119(e) to U.S. provisional patent application No. 63/332,397, filed Apr. 19, 2022, which is incorporated herein in its entirety.

### FIELD

This invention relates to methods of producing 3D-printed pharmaceutical products and resulting products.

### BACKGROUND OF THE INVENTION

Three-dimensional (3D) printing is becoming an attractive technology for the design and development of personalized dosage forms with improved palatability. Three-dimensional (3D) printing, as an evolving technology, has attracted a great deal of attention in various medical fields, particularly in drug delivery, such as fabrication of solid dosages forms (Scoutaris et al., 2018), films (Hafezi et al., 2019), and microneedles 20(Pere et al., 2018). Within the past decade, a significant number of published research have exploited 3D additive applications for the design and printing of novel drug delivery systems (Goyanes et al., 2015b; Scoutaris et al., 2016; Trenfield et al., 2018; Zhu et al., 2020). These studies have shown that the drug release can be tailored to each specific patient's need by adjusting the tablet design and the selection of compatible pharma grade polymers for 3D printing. The widely used 3D printing technologies include fused deposition modelling (FDM), selective laser sintering (SLS), stercolithography, and inkjet printing. Most of the 3D printing technologies are compatible with pharma grade polymers which makes 3D printing an attractive and alternative approach for the fabrication of solid dosage forms in particular for personalised medicine.

An advantage of FDM technology in comparison with other 3D printing technologies is it's compatibility of majority of pharma grade polymers such as hydroxypropyl methylcellulose (Kadry et al., 2018), hydroxypropyl methylcellulose acetate succinate (Goyanes et al., 2017), Ethyl cellulous (Yang et al., 2018) and Hydroxypropyl cellulose (Chai et al., 2017). FDM has been proved to be a versatile 3D printing technology in fabricating personalised medicines. Using the technology, tablets were printed with unique designs that resulted in modified drug release profiles (Goyanes et al., 2015a; Jamróz et al., 2018). Polypills (Pereira et al., 2019) and bilayer solid dosage forms (Ghanizadeh Tabriz et al., 2020; Gioumouxouzis et al., 2018) were also printed using FDM to improve patient's compliance (Castellano et al., 2014). SLS has also seen its application in fabrication of solid dosage forms. Fina et. al. studied the printability of several pharmaceutical grade polymers such as Eudragit, polyethylene oxide and ethyl cellulose using SLS technology (Fina et al., 2018, 2017). Wang et al (Wang et al., 2016) investigated the suitability of SLS technology in fabricating PEGDA/paracetamol tablets.

Martinez et. al. (Martinez et al., 2018) showed SLS may be used to fabricate drug loaded tablets with modified release by altering the tablet geometry. Inkjet printing has also shown its potential in fabricating of solid dosage forms despite there is limited choice of compatible polymers. A study by Kyobula et. al. (Kyobula et al., 2017)

### SUMMARY

The application relates to micro-extrusion based printing for the fabrication of pharmaceutical products. In aspect, the application relates to 3D ptinted tablets, for example, paediatric ibuprofen (IBU) chewable tablets. The application further relates to micro-extrusion based three-dimensional printing of tablets by assessing a range of front runner polymers, such as polymers that can provide taste masking and increase dissolution rates of water insoluble drugs. The printing fromulations comprise of drug/polymer powdered blends or granules. Furthermore, sweeteners and flavours can be included in the printing formualtions to improve taste and palatability. Due to the drug-polymer miscibility and the plasticization effect of many active pharmaceutical ingredients (API) micro-extrusion can be beneficial for processing taste masked tablets, especially, paediatric dosage forms. The printed tablets can result in high printing quality with high print reproducibilty and a smooth surface finish across the tablet's surface. Provided are micro-extrusion based methods for processing of powder blends containing active pharmaceutical ingredients for rapid printing and development of personalised dosage forms. The process allows the fabrication of tablets with different desings and active drug doses.

### DETAILED DESCRIPTION OF THE DRAWINGS

### Figures

FIG. 1: Schematic drawing of micro-extrusion printhead for tablet printing

FIG. 2: TGA graphs of bulk EP, soluplus, VA64 and ibuprofen

FIG. 3: Optical images of the 3D printed tablets based on 40% and 60% infill density. (a) EPO/IBU tablets, top view and (b) side view. (c) Soluplus/IBU tablets top and (d) side view. VA64/IBU tablets top, (f) side view, paediatric designs.

FIG. 4: SEM images of (a) EPO/IBU, (b) Soluplus/IBU and (c) VA64/IBU 3D printed tablets.

FIG. 5: XRD graphs of (a) plain EPO, Soluplus Ibuprofen. (b) physical mixtures of IBU/polymer formulations and the (c) the 3D printed tablets.

FIG. 6: DSC thermograms of bulk polymers and Ibuprofen

FIG. 7: Raman spectra of bulk polymers, Ibuprofen and 3D printed Ibuprofen/polymer tablets.

FIG. 8: (a) Explained variance of each PC for Ibu-S (left) and Ibu-VA64 sample, (b) comparison of first PC with the spectra of IBU, SOL and VA64 respectively, and (c) Concentration of chemical map for PC1 for Ibu-SOL and Ibu-VA64.

FIG. 9: Dissolution rates of (a) IBU-EPO, (b) IBU-VA64 and (c) IBU-SOL 3D printed tablets with different infill densities at pH 1.2 and pH 7.4 respectively.

FIG. 10: Dissolution profiles of IBU-EPO printed tablets with (a) 2.5% and 7.4% Neusilin (pH 1.2).

FIG. 11: Taste making evaluation of Ibuprofen, EPO, Soluplus, VA64 and 3D printed VA64/IBU, SOL/IBU and EPI/IBU tablets.

FIG. 12: Glass transition integrations of (a) Soluplus/IBU, (b) VA64/IBU and (c) EPO/IBU tablets.

The methods described herein can be used to prepare 3-D printed tablets containing a wide variety of tablets, preferably an API selected from one or more of: astemizole, azelastine, azatadine, brompheniramine, carbinoxamine, cetirizine, chlorpheniramine, clemastine, cyproheptadine, desloratadine, dexbrompheniramine, dexchlorpheniramine, diphenhydramine, fexofenadine, hydroxyzine, levocetirizine, loratadine, phenindamine, pheniramine, phenyltoloxamine, promethazine, pyrilamine, terfenadine, tripelennamine, triprolidine, acetyl dihydrocodeine, benproperine, benzonatate, benzylmorphine, bibenzonium bromide, butamirate, butorphanol, carbetapentane, chlophedianol, clobutinol, clofedanol, cloperastine, codeine, dextromethorphan, dextromethorphan hydrobromide, diacetylmorphine, dibunate, dihydrocodeine, dimemorfan, dimethoxanate, diphenhydramine, dropropizine, droxypropine, ethylmorphine, fedrilate, glaucine, hydrocodone, hydromorphone, isoaminile, laudanum, levodropropizine, levomethadone, levopropoxyphene, meprotixol, methadone, morclofone, nepinalone, nicocodine, nicodicodine, normethadone, noscapine, oxeladin, oxolamine, pentoxyverine, pholcodine, pipazetate, piperidione, prenoxdiazine, tipepidine, zipeprol, acetylcysteine, althea root, ambroxol, antimony pentasulfide, bromhexine, carbocisteine, cineole, combinations, combinations, creosote, dembrexine hydrochloride, domiodol, dornase alfa, eprazinone, erdosteine, guaiacolsulfonate, guaifenesin, hederae helicis folium, ipecacuanha, letosteine, levo verbenone, mannitol, mesna, neltenexine, potassium iodide, senega, sobrerol, stepronin, tiopronin, tyloxapol, pseudoephedrine, cetirizine, loratadine, fexofenadine, diphenhydramine, levocetirizine, desloratadine, phenol, ethanol, thymol, eucalyptol, ethanol, methyl salicylate, chlorhexidine gluconate, cetylpyridinium chloride, hexetidine, triclosan, hydrogen peroxide, domiphen bromide, bismuth subsalicylate, loperamide hydrochloride, cimetidine, famotidine, nizatidine, ranitidine, lansoprazole, omeprazole, esomeprazole, rabeprazole, pantoprazole, dexlansoprazole, diphenoxylate, dicyclomine, loperamide, rifaximin, alosetron, cholestyramine, linaclotide, lubiprostone, polycarbophil, psyllium, alclometasone, amcinonide, beclometasone, betamethasone, budesonide, ciclesonide, clobetasol, clobetasone, clocortolone, cloprednol, cortivazol, deflazacort, deoxycorticosterone, desonide desoximetasone, dexamethasone, diflorasone, diflucortolone, difluprednate, fluclorolone, fludrocortisone, fludroxycortide, flumetasone, flunisolide, fluocinolone acetonide, fluocinonide, fluocortin, fluocortolone, fluorometholone, fluperolone, fluticasone, fluticasone propionate, fluprednidene, formocortal, halcinonide, halometasone, hydrocortisone aceponate, hydrocortisone buteprate, hydrocortisone butyrate, loteprednol, medrysone, meprednisone, methylprednisolone, methylprednisolone aceponate, mometasone furoate, paramethasone, prednicarbate, prednisone, prednisolone, prednylidene, rimexolone, tixocortol, triamcinolone, ulobetasol, 5-fluorouracil, 5-fluorodeoxyuridine, capecitabine, calcium supplements, calcimimetics, cinacalcet, nicotine, nicotine polacrilex, bupropion, varenicline, disulfiram, calcium carbimide, acamprosate, naltrexone, buprenorphine, methadone, levacetylmethadol, lofexidine, betahistine, cinnarizine, flunarizine, acetylleucine, gangliosides, ganglioside derivatives, tirilazad, riluzole, xaliproden, hydroxybutyric acid, amifampridine, doxylamine, diphenhydramine hydrochloride, melatonin, 1-theanine, monofluorophosphate, lactoferrin, lysozyme, lactoperoxidase, glucose oxidase, mutanase, benzocaine, lidocaine, clove oil, sodium bicarbonate, citric acid, tartaric acid, aspirin, ibuprofen, aceclofenac, acemetacin, aloxiprin, azapropazone, benorilate, bromfenac, carprofen, celecoxib, choline magnesium salicylate, diclofenac, diflunisal, etodolac, etoricoxib, faislamine, fenbufen, fenoprofen, flurbiprofen, indometacin, ketoprofen, ketorolac, lornoxicam, loxoprofen, meloxicam, meclofenamic acid, mefenamic acid, meloxicam, metamizole, methyl salicylate, magnesium salicylate, nabumetone, naproxen, nimesulide, paracetamol, oxyphenbutazone, parecoxib, phenylbutazone, piroxicam, salicyl salicylate, sulindac, sulfinpyrazone, suprofen, tenoxicam, tiaprofenic acid, tolmetin, valdecoxib, acetylsalicylic acid, aloxiprin, aminophenazone, anilides, benorilate, benzomorphan derivatives, bezitramide, bucetin, buprenorphine, butorphanol, carbasalate calcium, choline salicylate, codeine, dextromoramide, dextropropoxyphene, dezocine, diamorphine, diflunisal, dihydrocodeine, dihydrocodone, dihydromorphine, diphenylpropylamine derivatives, dipyrocetyl, ethenzamide, fentanyl, floctafenine, flupirtine, glafenine, guacetisal, hydrocodone, hydrocodone bitartrate, hydromorphone, hydromorphone hydrochloride, imidazole salicylate, ketobemidone, metamizole sodium, methadone, morphinan derivatives, morphine, morphine sulphate pentahydrate, morphine-6-glucuronode, morpholine salicylate, nalbuphine, natural opium alkaloids, nefopam, nicomorphine, nifenazone, non-steroidal anti-inflammatory drugs (NSAID), norhydrocodone, noroxycodone, opioids, opium, oripavine derivatives, oxycodeine, oxycodone, oxycodone hydrochloride, oxymorphone, papaveretum, pentazocine, pethidine, phenacetin, phenazocine, phenazone, phenylpiperidine derivatives, piritramide, potassium salicylate, propacetamol, propyphenazone, pyrazolones, rimazolium, salicylamide, salicylic acid derivatives, salsalate, sodium salicylate, tapentadol, tilidine, tramadol, viminol, ziconotide, vitamin B12, cyanocobalamin, riboflavin, guarana, L-carnitine, vitamin A (retinol), B1 (thiamine), B2 (riboflavin), B complex, B6 (pyridoxine), B12 (cobalamin), C (ascorbic acid), D (cholecalciferol), E (tocopherol), F (linoleic acid), G, H (biotin), and K, and choline, folic acid, inositol, niacin, pantothenic acid, para-aminobenzoic acid, terpenoids (e.g., carotenoid terpenoids and non-carotenoid terpenoids), resveratrol, phytosterols, anthraquinones, capsaicin, chlorophyll, betaine, oxalic acid, acetyl-L-carnitine, allantoin, androstenediol, androstendione, betaine (trimethylglycine), calcium pyruvate (pyruvic acid), carnitine, carnosine, carotene, carotenoid, choline, chlorogenic acid, cholic acid, chondroitin sulfate, chondroitin sulfate, cholestan, chrysin, coenzyme Q10, conjugated linoleic acid, corosolic acid, creatine, dehydroepiandrosterone, dichlorophen, diindolymethane, dimethylglycine, dimercapto succinic acid, ebselen, ellagic acid, enzymes, fisetin, formononetin, glucaric acid (glucarate), glucosamine (HCl or sulfate), glucosamine (N-acetyl), glutathione, hesperidine, hydroxy-3-methylbutyric acid, 5-hydroxytryptophan, indole-3-carbinol, inositol, isothiocyanates, linolenic acid-gamma, lipoic acid (alpha), melatonin, methylsulfonylmethane, naringin, pancreatin, para-aminobenzoic acid, paraben (methyl or propyl), phenolics, phosphatidylcholine, phosphatidylserine, phytosterols, progesterone, pregnenolone, omega-3 fatty acids, quercetin, resveratrol, D-ribose, rutin, S-adenosylmethionine, salicylic acid, sulforaphane, tartaric acid, taxifolin, tetrahydropalmatine, theophyline, theobromine, tigogenin, troxerutin, or mixtures or combinations thereof.

Polymers such as ethyl cellulose, carboxymethyl cellulose, hydroxyethyl and hydroxypropyl celluloses, hydroxypropyl methyl cellulose, cellulose acetate phthalate, polyurethane, silicones polycarbonate, polychloroprene, polyisobutylene, polycyanoacrylate, poly (vinyl acetate), polystyrene, polypropylene, poly(vinyl chloride), polyethylene, poly (methyl methacrylate), poly (hydroxyethyl methacrylate), acrylic acid and butyl acrylate copolymer, 2-ethylhexyl acrylate and butyl acrylate copolymer, vinyl acetate and methyl acrylate copolymer, ethylene vinyl acetate and polyethylene terephthalate, ethylene vinyl acetate and polyethylene, polyethylene and polyethylene terephthalate, or mixtures or combinations thereof can be used for the methods described berein.

Polymers such as poly acrylic acid, poly ethylene oxide poly ethylene glycol, polyvinyl pyrrolidone, poly vinyl alcohol, polyacrylamide, poly isopropyl acrylamide and poly cyclopropyl methacrylamide, starch, sodium starch glycolate, (lactide-co-glycolide) polymers, aliginic acid, carrageenan chitosan, hyaluronic acid and pectinic acid, or mixtures or combinations thereof can be used for the methods described herein.

Polymers such as PVP/VA 64, PVP K12, PVP K15, PVP K30, PVP K60, PVP K90, PVP K120, Plasodone S630, Plasdone C12, Plasdone C15, Plasdone C17, Plasdone C30 HPMC-AS M. HPMCAS-L, HPMCAS-LMP, HPMCAS-HMP, HPMCAS-MMP, HPMCAS-LG, HPMCAS-MG, HPMCAS-HG, HPMCAS-HF, HPMCAS-MF, HPMCAS-LF, Eudragit L100-55, Eudragit S100, Eudragit RLPO, Eudragit RSPO, Eudragit L100, Eudragit EPO, HPMC E15, HPMC E3, HPMC K100, HPMC 4M. HPMC 100 LV, HPMC 15 LV HPMCP-HP55, PVA (Poly vinyl alcohol), and Soluplus can be used for the methods described herein.

Polyvinylpyrrolidone K30 (PVP K30), polyvinylpyrrolidone VA64 (PVP-VA64), hydroxypropyl cellulose L (HPC-L), hydroxypropyl methylcellulose E3 (HPMC E3), polyethylene caprolactam-polyvinyl acetate-polyethylene glycol graft copolymer (Soluplus) and polymethacrylate (Eudragit EPO) are preferably useful for tablet formation.

Descriptions of the polymers that are useful and listed above can be found in Martin's physical pharmacy and pharmaceutical sciences: physical chemical and biopharmaceutical principles in the pharmaceutical sciences.—6th ed./editor, Patrick J. Sinko (ISBN 978-0-7817-9766-5).

Sweeteners such as sucrose, sucralose, mannitol, xylitol, sorbitol, aspartame, accessulfame-K, cyclamate, saccharin or combinations can be used for the methods described herein

Flavors such as orange, strawberry, raspberry, lemon, vanilla, coffee, lime, caramel, toffee, chocolate, cherry, apple, grape or watemellon can be used for the methods described herein

The present invention may be embodied in other specific forms without departing from the spirit or essential attributes thereof. This invention encompasses all combinations of preferred aspects and/or embodiments of the invention noted herein. It is understood that any and all embodiments of the present invention may be taken in conjunction with any other embodiment or embodiments to describe additional more preferred embodiments. It is also to be understood that each individual element of the preferred embodiments is its own independent preferred embodiment. Furthermore, any element of an embodiment is meant to be combined with any and all other elements from any embodiment to describe an additional embodiment.

### EXAMPLES

### Materials

Soluplus (SOL) and PVP VA-64 (VA64) were kindly donated by BASF (BASF-Germany). Eudragit EPO (EPO) was kindly donated by Evonik (Evonik-Germany). Ibuprofen (IBU) was purchased from Sigma Aldrich (Sigma Aldrich, UK). Strawberry flavouring was purchased from Symrise Ltd. (Marlow, UK), sucralose was purchased from Merck Life Science UK Limited (Dorset, UK).

### Formulation Preparation

PVP-VA64, EPO or Soluplus were blended with Ibuprofen at a ratio of 60/40 (w/w) using a turbula shaker-mixer (Glen Mills T2F Shaker/Mixer, USA) at 100 rpm for ten minutes. Tablets were prepared with each blend using micro-extrusion technology.

### Thermal Gravimetric Analysis (TGA)

TGA technique (TGA Q5000 Thermal instruments, USA) was utilised to investigate the thermal stability of bulk polymers and Ibuprofen. 2-2.5 mg of polymer and drug samples were carefully weighed and positioned in to a standard aluminium pan. The samples were heated from 25° C. to 500° C. at 10° C./min. The extracted raw data were analysed via TA Universal Analysis software (Universal Analysis 2000, version 4.5A, TA instruments, USA).

Differential Scanning Calorimetry (DSC)

Differential scanning calorimeter (Mettler-Toledo 823e, Switzerland) was utilised to investigate the thermal behaviour of bulk materials and 3D printed tablets as well as for investigation of physical state of the Ibuprofen within the 3D printed tablets. bulk materials and a small portion of the 3D printed tablets were approximately weighed between 2-2.5 mg and located into a 40 μL aluminium pan and crimped promptly. The bulk polymer and 3D printed Samples were heated from 25° C. to 160° C. at a rate of 10° C./min. Ibuprofen was heated from 25° C. to 120° C. at a rate of 10° C./min. The extracted DSC thermograms of bulk materials and the 3D printed tablets were analysed through STARe Excellence Thermal Analysis Software (Mettler Toledo, Switzerland).

### Design and 3D Printing of Ibuprofen Tablets

Tablet designs were constructed to investigate the micro extrusion (printability) of various polymer/IBU formulations as shown in Table 1. The tablets were designed via SolidWorks software (Dassault Systems, USA) and converted into (stereolithography) stl files. Both tablets have the same diameter of 12 mm but different heights, (i.e., 3 mm or 2.4 mm) and were sliced with 40% and 60% infill density, respectively. The infill densities were designed in a manner to have a total weight of 250 mg comprising 100 mg of ibuprofen. A Bio-X (Celink, Sweden) bioprinter with a pneumatic thermoplastic printhead was used to 3D print tablets. Approximate 3 grams of each blend were placed into a heat-able metallic reservoir and the tablets were printed using a 0.4 mm nozzle, 0.4 mm layer height and 5 mm/s print speed.

The printing temperature for IBU/VA64, IBU/EPO and IBU/Soluplus was set to 120° C., 90° C. and 105° C. respectively. The respective pressure used for the formulations were set to 120 kPa, 110 kPa and 90 kPa while the build plate temperature was maintained at 15° C.

TABLE 1
3D printed IBU tablet formualtions for peadiatric dosage forms

| | IBU | | | EPO | XLT | NEU | SCR | Flavor |
|---|---|---|---|---|---|---|---|---|
| | (wt/wt %) | VA-64 | SOL | (wt/wt %) | (wt/wt %) | (wt/wt %) | (wt/wt %) | (wt/wt %) |
| F1 | 40 | 60 | | | | | | |
| F2 | 40 | | 60 | | | | | |
| F3 | 40 | | | 60.0 | | | | |
| F4 | 40 | | | 50.0 | 5 | 2.5 | 1 | 1.5 |
| F5 | 40 | | | 42.5 | 10 | 5.0 | 1 | 1.5 |
| F6 | 40 | | | 35.0 | 15 | 7.5 | 1 | 1.5 |

VA64: vinylpyrrolidone-vinyl acetate, EPO: Amino Methacrylate Copolymer, XLT: xylitol, NEU: Neusilin, SCR: sucralose

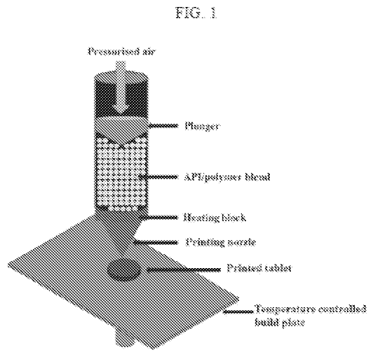

As shown in FIG. 1, the schematic drawing of micro-extrusion process of the tablets represents that a pressurised air pushes a metallic plunger towards the physical blend within the reservoir. By coordinating the horizontal movement of the printed head and the vertical movement of the printhead tablet designs with different densities were successfully fabricated.

### Scanning Electron Microscopy (SEM)

Scanning electron microscopy (Hitachi SU8030, Japan) was utilised to investigate the quality/layer height of the printed tablets via micro-extrusion. Tablets were kept secured on an aluminium stub with a conductive carbon adhesive tape (Agar Scientific, Stansted, UK). The tablets we then examined vis SEM and images were captured by an electron beam accelerating voltage of 1 KV and magnification of 30×.

### X-Ray Powder Diffraction (XRPD)

The physical state of the plain polymers, Ibuprofen and the 3D printed tablets (VA64/IBU, EPO/BU and Soluplus/IBU were investigated via XRPD. XRPD data were collected using a D8 Advance X-ray Diffractometer (Bruker AXS, Germany) equipped with a LynxEye silicon strip position sensitive detector and parallel beam optics. The diffractometer was operated with transmission geometry using Cu Kα radiation at 40 kV and 40 mA. The instrument was computer controlled using XRD commander software (Version 2.6.1, Bruker AXS, Germany) and the data was analysed using the EVA software (version 5.2.0.3, Bruker AXS, Germany). Samples were placed between foils of 2.5 μm thick mylar for measurement. Data was collected between 5-60° 2θ with a step size of 0.04° and a counting time of 0.2 seconds per step.

### In Vitro Dissolution and HPLC Analysis

In vitro dissolution studies were carried out to investigate the API release in acidic and neutral media for the 3D printed ibuprofen tablets. Drug release studies were carried out using a Varian 705 DS (USA) dissolution bath, attached with a paddle apparatus. Experiments were done at 37±1° C. using 800 mL of phosphate buffer saline (PBS, pH 7.4) in each vessel and paddles rotation were set to 100 rpm. PBS were prepared using 1.44 g disodium hydrogen phosphate, 0.24 g potassium dihydrogen phosphate and 8 g of sodium chloride (Barbero et al., 2016). Each time 2 mL of sample media were collected at 15, 30, 60, 90-and 120-minutes time interval and same amount of fresh PBS media were also added to maintain a constant volume of dissolution media. Collected samples were filtered using a 0.45 mm disk filter and poured into the High performance liquid chromatography vial for ibuprofen concentration analysis. This study was done using an Agilent 1200 series HPLC system equipped with a gradient elution system, an autosampler, HICHROM SSODS2-4889 (5×150×4 mm) column and an UV detector set at a wavelength of 214 nm. Samples were eluted using a mobile phase consisted of acetonitrile:water:Ortho-phosphoric acid (49.5:49.3:0.2 v/v) and pumped at a flow rate of 1.5 mL/min.

Such specifications showed approximately 115 bar of column back pressure with a retention time of 3 min. Calibration curve was also prepared using IBU in HPLC grade methanol at a concentration of 10, 20, 30, 40 and 50 μg/mL. All the experiments were studied in triplicates using the same method mentioned above.

### Taste Masking and Sensory Evaluation

In vivo taste masking investigation was caried out on 10 healthy human volunteers from whom informed consent was first obtained (approved by the Ethics Committee of the University of Greenwich). The study is also in accordance with the Code of Ethics of the World Medical Association (Declaration of Helsinki). The age category of the volunteers (either sex) was selected from a 18-30. The volunteers were trained and examined the bulk substance and the 3D printed tablets orally in the mouth for 2 minutes and spat out promptly. No substance and tablet were swallowed, and the moth was rinsed immediately after the experiment. The bitterness of the bulk substances and the 3D printed tablets were recorded from an intensity of scale of 1 to 5 where 1, 2, 3, 4, and 5 indicate no, threshold, slight, moderate, and strong bitterness.

The sensory training included a full description of the approach, proper use of the scale and confirmation that the samples prepared had sweetness levels that were perceived as “no sweet.”, “sweet”, “very sweet” and “extremely sweet” compared to the reference sample (equivalent sample of sucrose). Similarly, the strawberry aroma was assessed in terms of “sweet”, “strawberry”, “sour”, “fruity” and “aftertaste” attributes compared to a natural stripped strawberry juice. The panellist studies were conducted according to the World Medical Association's Code of Ethics (Declaration of Helsinki).

### Raman Spectra and Mapping

The Ibuprofen-based samples and its excipients were analysed using a Horiba LabRam I microscope fitted with a frequency-doubled Nd:YAG laser (λ=532 nm) with a 50 times long-working distance objective. Spectra of the IBU/EPO tablet and EPO polymer were recorded with a laser power of 25%, 8 accumulations at 1 second accumulation time; meanwhile a laser power of 25%, 4 accumulations at 2 second accumulations time was utilized for the remaining spectra. Finally, maps were generated over a 120×180 μm area with spectra acquired every 20 μm. Raman mapping was performed covering an area of 160×120 μm2 in a recorded of 20×20 spectra in x and y for Ibuprofen-EPO and Ibu-SOL samples while for IBU-VA64 33×25.

The spectra were initially baseline corrected using the asysmetric least square (AsLS) method having as parameters lambda equal to 105 and p equal to 10−3 ((Eilers and Boelens, 2005; R Core Team, 2019; Wehrens et al., 2015)). In order to correct the loss of focus and scattering effect due to abnormalities in tablets' surface, the spectra were normalized using the standard normal variate. Subsequently principal component analysis used to decompose the matrix to the most prevalent factors. The analysis was caried out using R programming language (R 4.03) (R Core Team 2019) and the ptw package to apply AsLS (Wehrens, Bloemberg, and Eilers 2015).

### Results

### Thermal Analysis Bulk Materials

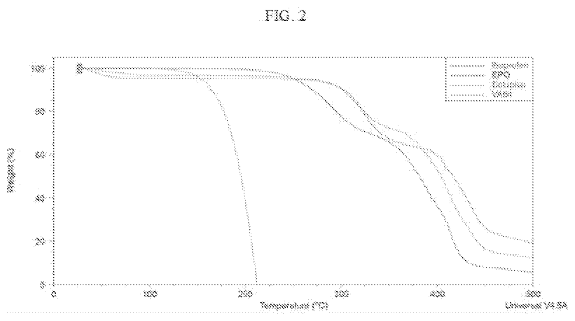

In order to identify the suitable micro-extrusion temperatures, TGA analysis was carried out to evaluate the thermal stability of IBU and the bulk polymers (FIG. 2). IBU was thermally stable when it was heated up to 140° C., beyond which the drug degraded with a rapid weight loss.

Soluplus (SOL) had a gradual 4% weight loss up to 90° C. due to removal of moisture and remained stable till 260° C. beyond which the polymer degraded. PVP VA64 (VA64) exhibited a slightly higher initial weight loss (5.5%) due to higher moisture content and did not have significant degradation till 260° C. Eudragit EPO (EPO) remained stable with no weight loss up to 220° C. followed by a rapid loss due to the polymer degradation.

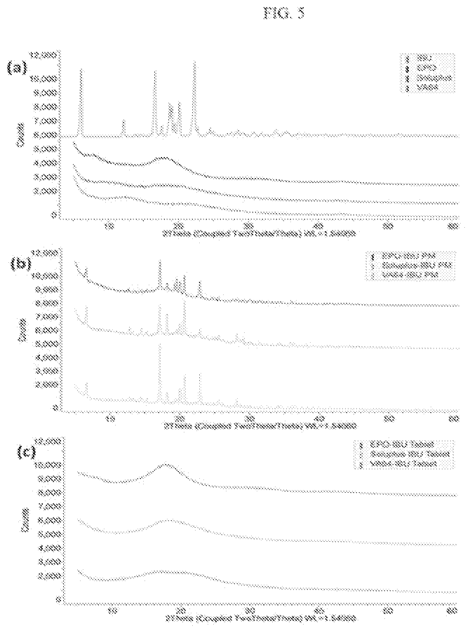

DSC thermal analysis was also carried out to characterize the thermal properties of IBU and the bulk polymers (FIG. 5a). IBU, as a crystalline solid, exhibited a sharp melting endotherm at 77.99° C. The bulk polymers, SOL, VA64, and EPO, exhibited glass transition temperature (Tg) at 66.33° C., 107.10° C., and 46.99° C., respectively. The change of heat capacity at the glass transition temperatures confirmed the amorphous nature of the polymers.

The operating temperature for the micro-extrusion should be maintained below the degradation temperature of each component. In addition, the drug/polymer blends may be processed around the glass transition temperatures of the polymers to facilitate the mixing of the drug and polymers. Therefore, 3D starting printing temprature of IBU/SOL, IBU/VA64, and IBU/EPO powder was carried out at 62.0° C., 105.0° C., and 46° C., respectively.

### Tablet Design and 3D Printing

Blends were printed into tablets with the same diameter of 10 mm and two infill densities (40% and 60%), to evaluate the printability and the impact of polymer type and infill density o n the drug release. All blends had an IBU/polymer ratio of 40/60 (w/w). With a total weight of 250 mg, each tablet consisted 100 mg of IBU. Previous studies demonstrated that VA64, SOL, and EPO were not suitable for 3D printing using Fusion Deposition Modelling (FDM) technology. The ductile nature of the polymers made it challenging to form filaments with the diameter and length desired for the following 3D printing (Alhijjaj et al., 2016; Fuenmayor et al., 2018; Sadia et al., 2016). High levels of plasticizers (e.g., 20%-30%) had to be added to the polymers to improve their printability, which limited the maximum drug load in the final tablets.

The major advantage of micro-extrusion technology is that drug/polymer blends may be printed directly without having to fabricating filaments as a pre-step. Using this method, a drug/polymer blend, at various ratios, is molten or soften in the cartridge. The soften drug/polymer blend is then extruded through a nozzle by applying pneumatic pressure (200-700 kPa). The resulting thermoplastic filament can be printed directly into tablets. Microextrusion technology, comparing to FDM and SLS, allows reduction of overall processing time of 3D printing and waste of printed materials. The latter is especially valuable when making personalized medicines consisting costly API's.

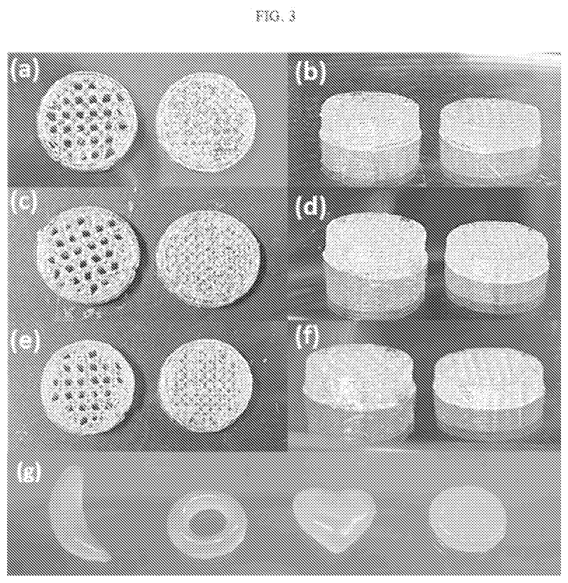

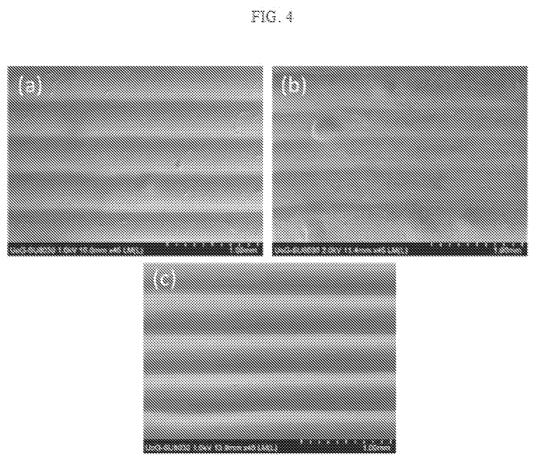

As shown in FIG. 3, tablets with both infill densities were successfully fabricated with good reproducibility using micro-extrusion 3D printing technique. SEM images of the printed tablets showed consistent height of deposited layers, which demonstrated the excellent print quality of the tablets (FIG. 4).

The speed of 3D printing depends on the size of nozzle, printing temperature, and pneumatic pressure applied to the blend. In this study, pneumatic pressures were maintained at 90-120 kPa with a 0.4 mm nozzle at the designated printing temperatures to give a print speed of approximately 5 mm/s. The print time of all three drug/polymer tablets was approximability 3 minutes. The temperature of the build bed was set at 10-40° C. to to allow tablets to be easily removed.

### Characterization of the Tablets

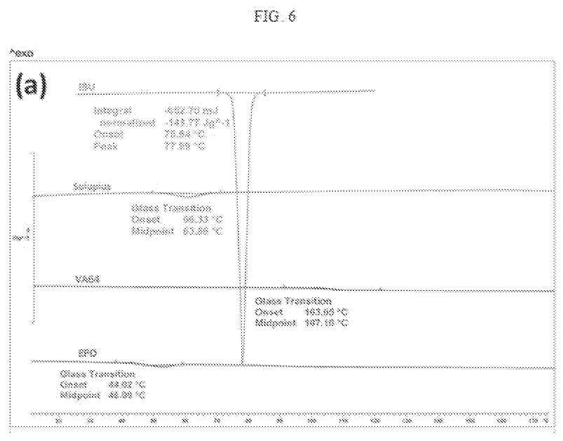

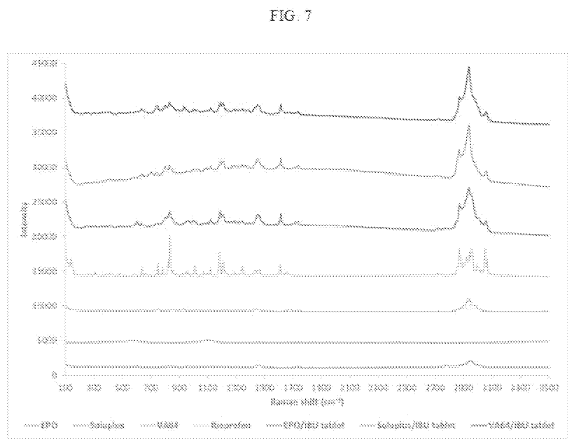

All three polymers used in this study are miscible with IBU at the drug level of 40% based on their solubility parameters (δ) (Islam et al., 2015; Maniruzzaman et al., 2015). X-ray powder diffractions analysis was used to evaluate the physical state of IBU within the 3D printed tablets. As shown in FIG. 6a, the XRPD patterns of the pure polymers (EPO, VA64 and SOL) featured broad halos indicating that all three polymers alone were in amorphous state. IBU solid exhibited its crystalline nature with its characteristic diffraction peaks at 5.8°, 12.2°, 16.1°, 17.4°, 18.5°, 20.0° and 22.2°/2θ. The characteristic XRPD patterns of IBU was also observed in the physical mixtures (IBU/EPO, IBU/SOL, IBU/VA64), which confirmed that the drug remained as crystalline solid prior to 3D printing. The characteristic diffraction peaks of IBU were not observed in the XRPD patterns of the 3D printed tablets, which suggested that IBU turned into amorphous state following microextrusion (FIG. 7c). XRPD pattern of the 3D printed tablets was also collected a week later. It was confirmed that IBU remained amorphous in the tablets stored at ambient conditions (25° C., 40% RH)

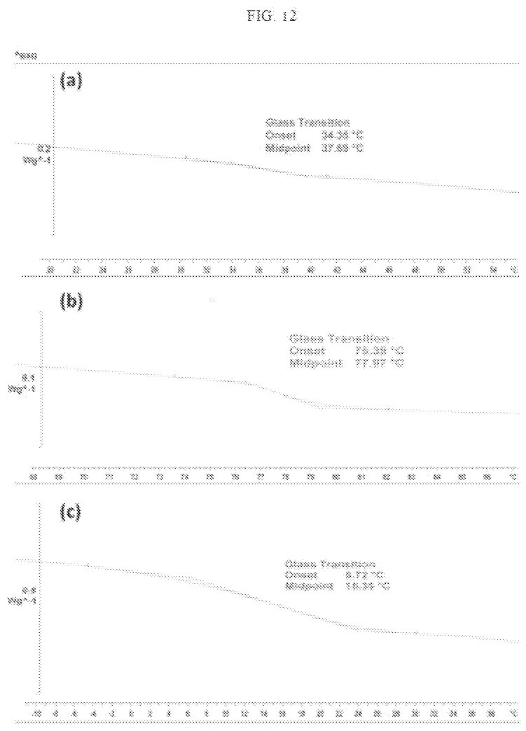

Thermal analysis was also carried out to investigate the physical state of IBU before and after 3D printing and possible drug-polymer interactions in the printed tablets. The bulk IBU exhibited a melting endotherm at 77.99° C. while SOL, VA 64 and EPO had glass transition (Tg) temperatures at 63.86° C., 107.10° C., and 46.99° C., respectively. The melting endotherm of IBU was not observed when the printed tablets were heated. As shown in FIG. 12, the IBU/SOL, IBU/VA64 and IBU/EPO thermograms exhibited single glass transitions at 37.69° C., 77.99° C. and 15.35° C., respectively. Both observations suggested that crystalline IBU was completely dissolved in the polymers at a drug load of 40% following 3D printing. Amorphous IBU, with a glass transition temperature of −45.15° C. (Dudognon et al., 2008), acted as a plasticizer in the printed tablets and led to lower Tg's [(Gryczke et al., 2011; Islam et al., 2015; Maniruzzaman et al., 2012). Interestingly, the measured glass transition temperatures were higher than the values predicted based on Fox equation. Fox equation has been widely used to estimate the glass transition temperatures of physical mixtures without interaction between the components. This discrepancy suggested strong intramolecular interactions between IBU and the studied polymers.

### Raman Spectra and Mapping

The interaction between IBU and the polymers were further investigated using Raman spectroscopy. The vibrational bands in the Raman spectra of IBU have been well documented in recent literatures (Breitenbach et al., 1999; Vueba et al., 2008). As shown in FIG. 7, crystalline IBU features hydroxyl methyl and aromatic peaks and more specifically the peak at 637 cm⁻¹ represents the out of plane stretching of CO—H bond. Other peaks at 746 cm⁻¹, 850 cm⁻¹, 1609 cm⁻¹, and 3047 cm⁻¹ correspond to the out-of-plane deformation of the C—H bonds, the out-of-plane bending the C—H bonds, the stretching of the C—C bonds, and the stretching of the C—H bonds, respectively. In addition, the peaks at 1181 cm⁻¹, 1452 cm⁻¹, and 2965 cm⁻¹ are related to the stretching of non-aromatic C—C bond, the asymmetric deformation of CH₃, and the asymmetric stretching of CH₃ bonds, respectively. The same bands were observed in the Raman spectra of the 3D printed IBU/polymer tablets (FIG. 7) but with broadened figures comparing to IBU alone.

As shown in FIG. 7, the peaks of C═C vibration of the aryl group in IBU shifted from 1608 cm⁻¹ to 1616 cm⁻¹ after 3D printed together with the polymers, which suggested a potential H-bond between IBU and the polymers (Hedoux et al., 2011). In addition, the C═O stretching of IBU shifted from 1652 cm−1 in the spectra of IBU alone to 1734 cm⁻¹ in that of the printed tablets, which is associated to and suggests a strong drug-polymer interaction similar to the one observed for cyclodextrin molecular complexes (Brás et al., 2008). It was also observed that the CO—H out-of-plane bending of the bulk IBU at 637 cm⁻¹ and 663 cm⁻¹ shifted to 605 cm⁻¹ or completely disappeared in the 3D printed tablets (Vueba et al., 2008). This shift suggested a static disorder molecular environment that resulted from the formation of a glass solutions.

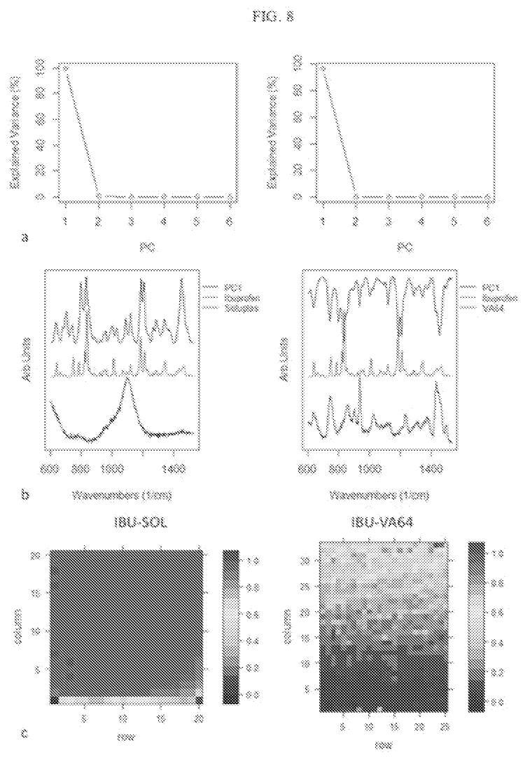

In order to investigate the spatial resolution of IBU in the printed tablet the Raman spectra were continuously recorded. PCA has been applied to decompose the spectra into scores and loadings that approximately express the initial spectra. The main advantage of PCA is that it does not require the use of the spectra of bulk compounds which could be undergo changes during print processing due to molecular interactions. The number of principal components needed to decompose the hyperspectral matrix is usually determined by the analysis of eigenvalues which expresses the total variance captured by a specified principal component. In simple systems such as in this case where only two compounds are present with good mixing, two-three PCs are adequate to decompose the system. In more complicated systems the use of more PCs, where a compound can be identified in a very late component, is not uncommon (Scoutaris et al. 2014). FIG. 8a illustrates the cumulative variance that corresponds to each PC. In the IBU-SOL 3D printed sample the PC (left) expresses 99.4% of the total variance whereas in the of Ibu-VA64, the PC (right) covers 96.7% of the total variance. For the former sample the very high variance covered by the PC is due to the very broad peaks of SOL spectrum which cannot be distinguished from that of IBU.

FIG. 8b represents a summary of the PC and the spectra of the tablet's individual components. For the IBU-VA64 sample it is evident that the PC contains peaks corresponding to both components namely the API and the polymer. However, this is not the case for the IBU-SOL samples which couldn't be distinguished due to the presence of its broad peak. As shown in FIG. 8c the plotting of the concentration map for the first PC of the Ibu-SOL and the Ibu-VA64 demonstrates that the compounds are homogeneously distributed within the tablet. This is a proof of strong drug-polymer intermolecular interactions and that the IBU is molecularly dispersed within the polymer matrix.

### Dissolution Studies

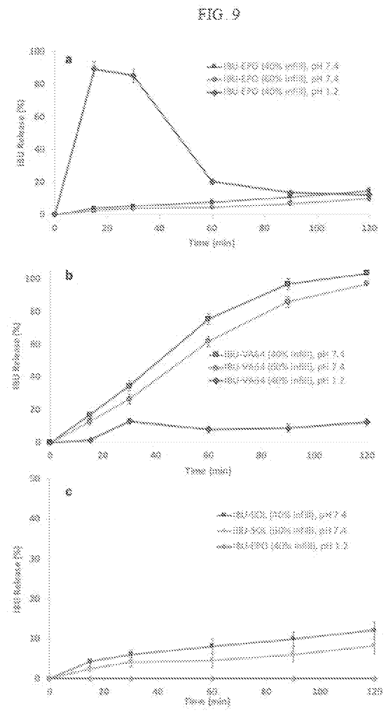

Drug release may be modified by dispersing the drug into a polymeric matrix at molecular level following 3D printing of a drug/polymer blend (Douroumis et al., 2007; Islam et al., 2015; Maniruzzaman et al. 2015) The dissolutions of IBU from the 3D printed tablets were evaluated in both acidic (pH 1.2) and alkaline media (pH 7.4) and revealed the impact of polymer type as well as the infill density (40 and 60%) on the drug release from the tablets. Eudragit EPO is a basic methacrylate copolymer that has been commonly used in immediate elease drug product requiring taste masking because it is practically insoluble at pH greater than 5 (in saliva), while highly soluble in acidic media (in stomach). As expected, IBU/EPO tablets exhibited a slow dissolution at pH 7.4, despite IBU was dispersed in the polymer matrix at the molecular level (Fine-Shamir and Dahan, 2019; Gryczke et al., 2011). The tablet with 40% infill density showed an ˜15% drug release in 2 hours, while the table with 60% infill density presented an ˜10% drug release in the same period (FIG. 9a). A slightly higher drug release from the tablet with lower fill density was attributed to larger surface of the tablet. The IBU/EPO tablets had a rapid dissolution at pH 1.2 with ˜89% drug released within 15 minutes and generated a high degree of supersaturation. As EPO was not a good crystallization inhibitor the concentration of IBU in the dissolution media dropped significantly following the initial drug release due to the crystallization of IBU. PVP VA 64 is a neutral vinylpyrrolidone-vinyl acetate copolymer with good solubility in water across large pH range. IBU is an acidic compound (pKa 4.6), which has a low solubility at low pH. The release of IBU from the 3D printed IBU/VA64 tablets were found low at pH 1.2 with an ˜13% drug release in 2 hours (FIG. 9b). The slow release of IBU from VA64 matrix with high drug load at low pH □ 2 was also observed previously (Tres et al., 2016). The authors concluded that VA64 dissolved preferentially from the exterior of the printed tablet (compact) that resulted in an amorphous IBU-rich and hydrophobic shell. The hydrophobic shell acted as in situ enteric coating and inhibits the dissolution. At pH 7.4, the solubility of IBU was significantly higher, which had IBU released at a similar rate as polymer. The congruency of IBU and polymer generated a supersaturation of IBU. In addition, PVP VA64 inhibited the crystallization of IBU in the solution, which maintained the level of supersaturation throughout the dissolution test. The IBU/VA64 tablet with 40% fill density showed a slightly higher dissolution rate than the tablet with 60% fill density. This comparison again confirmed that greater tablet surface resulted in higher drug release.

Soluplus is a well-known polymeric solubilizer that was found to facilitate drug release in several occasions due to the enhanced drug-polymer interactions. However, as shown in FIG. 9c, all IBU/SOL tablets exhibited slow dissolution at both pH 1.2 and 7.4. Pudlas et al. had a similar observation for IBU/SOL extrudates and investigated the root cause using spectroscopic analysis. The slow release of IBU was attributed to the strong H-bonding between IBU and VA64 which consumed large number of hydrophilic groups in the polymer chain thus significantly reduced the hydrophilicity of the polymer.

Comparatively, the drug release at pH 1.2 was slowest due to the low solubility of IBU at acidic conditions. The impact of infill density on the drug release was again observed, i.e., the IBU/Sol tablet with 40% fill density gave slightly faster dissolution than that with 60% fill density. Overall, the tablets with lower infill density gave a higher dissolution rate due to higher surface area. The impact of polymer on the dissolution of the IBU/polymer tablets depends on the characteristics of the compound, polymers, as well as their interactions.

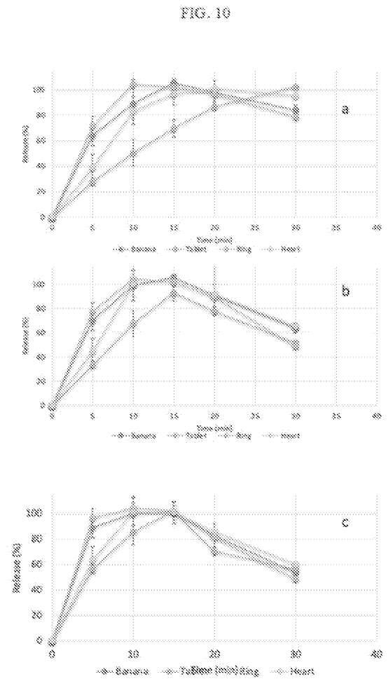

Furthermore, dissolution studies were conducted for F4-F6 with the addition of sweeteners (e.g., xylitol or combinations thereof) and inorganic excipients (magnesium aluminometacilicates) combined with IBU/EPO. The results (FIG. 10) showed rapid IBU dissolution rates within the first 5-10 min. The dissolution rates were affected by the shape of the printed tablets (e.g., ring>banana>heart) due to the different surface area.

### Taste Masking Evaluation

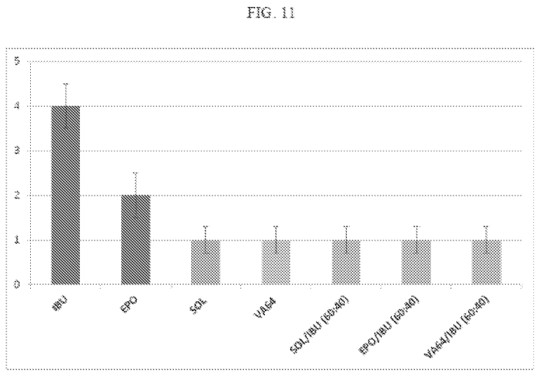

A drug product for paediatric use is governed by stricter regulations introduced by the European Medicines Agency (EMEA, 2007). Strickley et al. (2008) identified challenges in paediatric product development associated to dose flexibility and taste masking. IBU is known to have a bitter or salty taste and thus taste masking is required in the finished product consisting of IBU for paediatric use. As shown in FIG. 11, the taste of IBU and the 3D printed IBU/polymer tablets were assessed in healthy panellists. IBU had an average score of 4, which confirmed the bitterness taste of the drug while the bulk polymers had acceptable taste with bitterness scores of 2 or below. All 3D printed IBU/polymer tablets had pleasant taste with bitterness score of 1. As previously reported the effective taste masking of bitter APIs is directly related to the H-bonding interactions between the drug and polymer carriers (Gryczke et al., 2011, Maniruzzaman 2012). The effective taste masking of IBU in the chewable tablets demonstrated that micro-extrusion can be successfully used for 3D printing of personalized tablets for paediatric use.

The study demonstrated a simple micro-extrusion 3D printing technique that can be used to 3D print tablets suitable and reproducible printability. Such a 3D printing technology can bypass the pre-and post-processing procedures that other 3D printing technologies require. In addition, such an approach minimises material wastes compared to other 3D printing technologies. Similar to other 3D printing technology, micro-extrusion 3D printing can also be used to fabricate drug products with specific release profile tailored to individual patient by selecting polymer matrix and modifying infill density of the tablets.

Due to the drug-polymer interactions induced during printing processing, ibuprofen (IBU) was found to form a glass solution confirmed by differential calorimetry (DSC) while H-bonding interactions were identified by confocal Raman mapping. IBU was also found to be uniformly distributed within the polymer matrices at molecular level. The tablet palatability was assessed by panellists and revealed excellent taste masking of the IBU's bitter taste. Overall micro-extrusion demonstrated promising processing capabilities of powder blends for rapid printing and development of personalised dosage forms.

The sensory analysis of 3D printed tablets involved their evolution in terms of sweetness intensity and strawberry/orange arom. Several studies have shown possible interactions between the sweeteners and flavours which ultimately alter the sweetener potency]. A critical consideration in such studies is to identify the optimal ratio of sweetener/flavour including their total concertation in the dosage form. Herein, the optimized Sucralose/strawberry and aspartame/orange ratio was 0.9:1.1 wt/wt % and 1.0/1.5 wt/wt % while several other ratios were tested.

Tables 2-3 show that the bulk sucralose and aspartame presented very to extremely sweet intensity respectively and a very strong aftertaste. For the 3D printed tablets, the subjects scored reduced intensity and most of them reported “very sweet” while a few “moderate sweet” for both flavours. For the perceive aftertaste the subjects scored reduced sweet aftertaste in comparison to the bulk sucralose and aspartame.

Likewise, for the strawberry and orange aroma evaluation of the bulk powders the subjects scored a strong strawberry/orange and fruit intensity. Interestingly, the evaluation of the 3D tablets revealed a strong sweet, strawberry (or orange), fruity intensity and most of the subjects scored strong aftertaste. The outcomes are related to the synergistic interactions between the sweetener and the flavours where the total intensity of the mixture is greater than that of the individual excipients. Furthermore, the flavor (strawberry/orange) and fruity perception of the 3D tablets were not affected and remained strong despite the presence of other excipients in the formulation. Overall, the optimized sweetener/flavour ratios provided excellent taste perception with superior.

TABLE 2
Evaluation of sweetness and strawberry aroma of
bulk substances and 3D printed fruit chews.

| | Sweetness | | | | |
|---|---|---|---|---|---|
| | No | Moderate | Very | Extremely | |
| | Sweet | Sweet | Sweet | Sweet | Aftertaste |
| Sucralose | — | — | 1-2 | 8-10 | 8-10 |
| 3D tablets | — | 1-2 | 8-10 | — | 6-8 |

| | Strawberry Aroma | | | | |
|---|---|---|---|---|---|
| | Sweet | Strawberry | Sour | Fruity | Aftertaste |
| Strawberry | 6-8 | 9-10 | — | 8-10 | 5-7 |
| 3D tablet | 8-10 | 9-10 | — | 8-10 | 8-10 |

TABLE 3
Evaluation of sweetness and orange aroma of
bulk substances and 3D printed fruit chews.

| | Sweetness | | | | |
|---|---|---|---|---|---|
| | No | Moderate | Very | Extremely | |
| | Sweet | Sweet | Sweet | Sweet | Aftertaste |
| Aspartame | — | 1-2 | 8-10 | | 8-10 |
| 3D tablets | — | 1-2 | 8-10 | — | 6-8 |

| | Orange Aroma | | | | |
|---|---|---|---|---|---|
| | Sweet | Orange | Sour | Fruity | Aftertaste |
| Orange | 6-8 | 9-10 | — | 8-10 | 5-7 |
| 3D tablet | 8-10 | 9-10 | — | 8-10 | 8-10 |

### References

Alhijjaj, M., Belton, P., Qi, S., 2016. An investigation into the use of polymer blends to improve the printability of and regulate drug release from pharmaceutical solid dispersions prepared via fused deposition modeling (FDM) 3D printing. Eur. J. Pharm. Biopharm. 108, 111-125. https://doi.org/https://doi.org/10.1016/j.ejpb.2016.08.016

Barbero, N., Barolo, C., Viscardi, G., 2016. Bovine Serum Albumin Bioconjugation with FITC. World J. Chem. Educ. 4, 80-85. https://doi.org/10.12691/wjce-4-4-3

Brás, A. R., Noronha, J. P., Antunes, A. M. M., Cardoso, M. M., Schönhals, A., Affouard, F., Dionísio, M., Correia, N. T., 2008. Molecular Motions in Amorphous Ibuprofen As Studied by Broadband Dielectric Spectroscopy. J. Phys. Chem. B 112, 11087-11099. https://doi.org/10.1021/jp8040428

Breitenbach, J., Schrof, W., Neumann, J., 1999. Confocal Raman-Spectroscopy: Analytical Approach to Solid Dispersions and Mapping of Drugs. Pharm. Res. 16, 1109-1113. https://doi.org/10.1023/A: 1018956304595

Castellano, J. M., Sanz, G., Peñalvo, J. L., Bansilal, S., Fernández-Ortiz, A., Alvarez, L., Guzmán, L., Linares, J. C., García, F., D'Aniello, F., Arnáiz, J. A., Varca, S., Martínez, F., Lorenzatti, A., Imaz, I., Sánchez-Gómez, L. M., Roncaglioni, M. C., Baviera, M., Smith, S. C., Taubert, K., Pocock, S., Brotons, C., Farkouh, M. E., Fuster, V., 2014. A Polypill Strategy to Improve Adherence: Results From the FOCUS Project. J. Am. Coll. Cardiol. 64, 2071-2082. https://doi.org/https://doi.org/10.1016/j.jacc.2014.08.021

Chai, X., Chai, H., Wang, X., Yang, J., Li, J., Zhao, Y., Cai, W., Tao, T., Xiang, X., 2017. Fused Deposition Modeling (FDM) 3D Printed Tablets for Intragastric Floating Delivery of Domperidone. Sci. Rep. 7, 2829. https://doi.org/10.1038/s41598-017-03097-x

Douroumis, D., Bouropoulos, N., Fahr, A., 2007. Physicochemical characterization of solid dispersions of three antiepileptic drugs prepared by solvent evaporation method. J. Pharm. Pharmacol. 59, 645-653. https://doi.org/https://doi.org/10.1211/jpp.59.5.0004

Dudognon, E., Danède, F., Descamps, M., Correia, N. T., 2008. Evidence for a New Crystalline Phase of Racemic Ibuprofen. Pharm. Res. 25, 2853-2858. https://doi.org/10.1007/s11095-008-9655-7

Eilers, P. H. C., Boelens, H. F. M., 2005. Baseline Correction with Asymmetric Least Squares Smoothing. Life Sci.

Elbadawi, M., Gustaffson, T., Gaisford, S., Basit, A. W., 2020. 3D printing tablets: Predicting printability and drug dissolution from rheological data. Int. J. Pharm. 590, 119868. https://doi.org/https://doi.org/10.1016/j.ijpharm.2020.119868

Fina, F., Goyanes, A., Gaisford, S., Basit, A.W., 2017. Selective laser sintering (SLS) 3D printing of medicines. Int. J. Pharm. 529, 285-293. https://doi.org/https://doi.org/10.1016/j.ijpharm.2017.06.082

Fina, F., Goyanes, A., Madla, C. M., Awad, A., Trenfield, S. J., Kuek, J. M., Patel, P., Gaisford, S., Basit, A. W., 2018. 3D printing of drug-loaded gyroid lattices using selective laser sintering. Int. J. Pharm. 547, 44-52. https://doi.org/https://doi.org/10.1016/j.ijpharm.2018.05.044

Fine-Shamir, N., Dahan, A., 2019. Methacrylate-Copolymer Eudragit EPO as a Solubility-Enabling Excipient for Anionic Drugs: Investigation of Drug Solubility, Intestinal Permeability, and Their Interplay. Mol. Pharm. 16, 2884-2891. https://doi.org/10.1021/acs.molpharmaceut.9b00057

Fuenmayor, E., Forde, M., Healy, A. V, Devine, D. M., Lyons, J. G., McConville, C., Major, I., 2018. Material Considerations for Fused-Filament Fabrication of Solid Dosage Forms. Pharmaceutics https://doi.org/10.3390/pharmaceutics10020044

Ghanizadeh Tabriz, A., Nandi, U., Hurt, A. P., Hui, H.-W., Karki, S., Gong, Y., Kumar, S., Douroumis, D., 2020. 3D Printed Bilayer Tablet with Dual Controlled Drug Release for Tuberculosis Treatment. Int. J. Pharm. 120147. https://doi.org/https://doi.org/10.1016/j.ijpharm.2020.120147

Gioumouxouzis, C. I., Baklavaridis, A., Katsamenis, O. L., Markopoulou, C. K., Bouropoulos, N., Tzetzis, D., Fatouros, D. G., 2018. A 3D printed bilayer oral solid dosage form combining metformin for prolonged and glimepiride for immediate drug delivery. Eur. J. Pharm. Sci. 120, 40-52. https://doi.org/https://doi.org/10.1016/j.ejps.2018.04.020

Goyanes, A., Buanz, A. B. M., Hatton, G. B., Gaisford, S., Basit, A. W., 2015a. 3D printing of modified-release aminosalicylate (4-ASA and 5-ASA) tablets. Eur. J. Pharm. Biopharm. 89, 157-162. https://doi.org/https://doi.org/10.1016/j.ejpb.2014.12.003

Goyanes, A., Fina, F., Martorana, A., Sedough, D., Gaisford, S., Basit, A. W., 2017. Development of modified release 3D printed tablets (printlets) with pharmaceutical excipients using additive manufacturing. Int. J. Pharm. 527, 21-30. https://doi.org/https://doi.org/10.1016/j.ijpharm.2017.05.021

Goyanes, A., Wang, J., Buanz, A., Martínez-Pacheco, R., Telford, R., Gaisford, S., Basit, A. W., 2015b. 3D Printing of Medicines: Engineering Novel Oral Devices with Unique Design and Drug Release Characteristics. Mol. Pharm. 12, 4077-4084. https://doi.org/10.1021/acs.molpharmaceut.5b00510

Gryczke, A., Schminke, S., Maniruzzaman, M., Beck, J., Douroumis, D., 2011. Development and evaluation of orally disintegrating tablets (ODTs) containing Ibuprofen granules prepared by hot melt extrusion. Colloids Surf. B. Biointerfaces 86, 275-284. https://doi.org/10.1016/j.colsurfb.2011.04.007

Hafezi, F., Scoutaris, N., Douroumis, D., Boateng, J., 2019. 3D printed chitosan dressing crosslinked with genipin for potential healing of chronic wounds. Int. J. Pharm. 560, 406-415. https://doi.org/https://doi.org/10.1016/j.ijpharm.2019.02.020

Hédoux, A., Guinet, Y., Derollez, P., Dudognon, E., Correia, N. T., 2011. Raman spectroscopy of racemic ibuprofen: Evidence of molecular disorder in phase II. Int. J. Pharm, 421, 45-52. https://doi.org/https://doi.org/10.1016/j.ijpharm.2011.09.015

Islam, M. T., Scoutaris, N., Maniruzzaman, M., Moradiya, H. G., Halsey, S. A., Bradley, M. S. A., Chowdhry, B. Z., Snowden, M. J., Douroumis, D., 2015. Implementation of transmission NIR as a PAT tool for monitoring drug transformation during HME processing. Eur. J. Pharm. Biopharm. Off. J. Arbeitsgemeinschaft fur Pharm. Verfahrenstechnik e.V 96, 106-116. https://doi.org/10.1016/j.ejpb.2015.06.021

Jamróz, W., Kurek, M., Czech, A., Szafranicc, J., Gawlak, K., Jachowicz, R., 2018. 3D printing of tablets containing amorphous aripiprazole by filaments co-extrusion. Eur. J. Pharm. Biopharm. 131, 44-47. https://doi.org/https://doi.org/10.1016/j.ejpb.2018.07.017

Januskaite, P., Xu, X., Ranmal, S. R., Gaisford, S., Basit, A. W., Tuleu, C., Goyanes, A., 2020. I Spy with My Little Eye: A Paediatric Visual Preferences Survey of 3D Printed Tablets. Pharmaceutics 12. https://doi.org/10.3390/pharmaceutics12111100

Kadry, H., Al-Hilal, T. A., Keshavarz, A., Alam, F., Xu, C., Joy, A., Ahsan, F., 2018. Multipurposable filaments of HPMC for 3D printing of medications with tailored drug release and timed-absorption. Int. J. Pharm. 544, 285-296. https://doi.org/10.1016/J.IJPHARM.2018.04.010

Kyobula, M., Adedeji, A., Alexander, M. R., Saleb, E., Wildman, R., Ashcroft, I., Gellert, P. R., Roberts, C. J., 2017. 3D inkjet printing of tablets exploiting bespoke complex geometries for controlled and tuneable drug release. J. Control. Release 261, 207-215. https://doi.org/https://doi.org/10.1016/j.jconrel.2017.06.025

Maniruzzaman, M., Boateng, J. S., Bonnefille, M., Aranyos, A., Mitchell, J. C., Douroumis, D., 2012. Taste masking of paracetamol by hot-melt extrusion: An in vitro and in vivo evaluation. Eur. J. Pharm. Biopharm. 80, 433-442. https://doi.org/https://doi.org/10.1016/j.ejpb.2011.10.019

Maniruzzaman, M., Pang, J., Morgan, D. J., Douroumis, D., 2015. Molecular Modeling as a Predictive Tool for the Development of Solid Dispersions. Mol. Pharm. 12, 1040-1049. https://doi.org/10.1021/mp500510m

Martinez, P. R., Goyanes, A., Basit, A. W., Gaisford, S., 2018. Influence of Geometry on the Drug Release Profiles of Stereolithographic (SLA) 3D-Printed Tablets. AAPS PharmSciTech 19, 3355-3361. https://doi.org/10.1208/s12249-018-1075-3

Nasereddin, J. M., Wellner, N., Alhijjaj, M., Belton, P., Qi, S., 2018. Development of a Simple Mechanical Screening Method for Predicting the Feedability of a Pharmaceutical FDM 3D Printing Filament. Pharm. Res. 35, 151. https://doi.org/10.1007/s11095-018-2432-3

Öblom, H., Zhang, J., Pimparade, M., Speer, I., Preis, M., Repka, M., Sandler, N., 2019. 3D-Printed Isoniazid Tablets for the Treatment and Prevention of Tuberculosis—Personalized Dosing and Drug Release. AAPS PharmSciTech 20, 52. https://doi.org/10.1208/s12249-018-1233-7

Pere, C. P. P., Economidou, S. N., Lall, G., Ziraud, C., Boateng, J. S., Alexander, B. D., Lamprou, D. A., Douroumis, D., 2018. 3D printed microneedles for insulin skin delivery. Int. J. Pharm. 544, 425-432. https://doi.org/https://doi.org/10.1016/j.jjpharm.2018.03.031

Pereira, B. C., Isreb, A., Forbes, R. T., Dores, F., Habashy, R., Petit, J. -B., Alhnan, M. A., Oga, E. F., 2019. ‘Temporary Plasticiser’: A novel solution to fabricate 3D printed patient-centred cardiovascular ‘Polypill’ architectures. Eur. J. Pharm. Biopharm. 135, 94-103. https://doi.org/https://doi.org/10.1016/j.ejpb.2018.12.009

R Core Team, 2019. R: A language and environment for statistical computing. R Found. Stat. Comput.

Rycerz, K., Stepien, K. A., Czapiewska, M., Arafat, B. T., Habashy, R., Isreb, A., Peak, M., Alhnan, M. A., 2019. Embedded 3D Printing of Novel Bespoke Soft Dosage Form Concept for Pediatrics. Pharmaceutics 11, 630. https://doi.org/10.3390/pharmaceutics11120630

Sadia, M., Sośnicka, A., Arafat, B., Isreb, A., Ahmed, W., Kelarakis, A., Alhnan, M. A., 2016. Adaptation of pharmaceutical excipients to FDM 3D printing for the fabrication of patienttailored immediate release tablets. Int. J. Pharm. 513, 659-668. https://doi.org/https://doi.org/10.1016/j.ijpharm.2016.09.050

Scoutaris, N., Ross, S., Douroumis, D., 2016. Current Trends on Medical and Pharmaceutical Applications of Inkjet Printing Technology. Pharm. Res. 33, 1799-1816. https://doi.org/10.1007/s11095-016-1931-3

Scoutaris, N., Ross, S. A., Douroumis, D., 2018. 3D Printed “Starmix” Drug Loaded Dosage Forms for Paediatric Applications. Pharm. Res. 35, 34. https://doi.org/10.1007/s11095-017-2284-2

Strickley, R. G., Iwata, Q., Wu, S., Dahl, T. C., 2008. Pediatric drugs—a review of commercially available oral formulations. J. Pharm. Sci. 97, 1731-1774. https://doi.org/10.1002/jps.21101

Tagami, T., Ito, E., Kida, R., Hirose, K., Noda, T., Ozeki, T., 2021. 3D printing of gummy drug formulations composed of gelatin and an HPMC-based hydrogel for pediatric use. Int. J. Pharm. 594, 120118. https://doi.org/https://doi.org/10.1016/j.ijpharm.2020.120118

Trenfield, S. J., Awad, A., Goyanes, A., Gaisford, S., Basit, A. W., 2018. 3D Printing Pharmaceuticals: Drug Development to Frontline Care. Trends Pharmacol. Sci. 39, 440-451. https://doi.org/https://doi.org/10.1016/j.tips.2018.02.006

Tres, F., Treacher, K., Booth, J., Hughes, L. P., Wren, S. A. C., Aylott, J. W., Burley, J. C., 2016. Indomethacin-Kollidon VA64 Extrudates: A Mechanistic Study of pH-Dependent Controlled Release. Mol. Pharm. 13, 1166-1175, https://doi.org/10.1021/acs.molpharmaceut.5b00979

Vueba, M. L., Pina, M. E., Batista de Carvalho, L. A. E., 2008. Conformational Stability of Ibuprofen: Assessed by DFT Calculations and Optical Vibrational Spectroscopy. J. Pharm. Sci. 97, 845-859. https://doi.org/https://doi.org/10.1002/jps.21007

Wang, H., Dumpa, N., Bandari, S., Durig, T., Repka, M. A., 2020, Fabrication of Taste-Masked Donut-Shaped Tablets Via Fused Filament Fabrication 3D Printing Paired with Hot-Melt Extrusion Techniques. AAPS Pharm SciTech 21, 243. https://doi.org/10.1208/s12249-020-01783-0

Wang, J., Goyanes, A., Gaisford, S., Basit, A. W., 2016. Stereolithographic (SLA) 3D printing of oral modified-release dosage forms. Int. J. Pharm. 503, 207-212. https://doi.org/https://doi.org/10.1016/j.ijpharm.2016.03.016

Wehrens, R., Bloemberg, T. G., Eilers, P. H. C., 2015. Fast parametric time warping of peak lists. Bioinformatics. https://doi.org/10.1093/bioinformatics/btv299

Xu, P., Li, J., Meda, A., Osei-Yeboah, F., Peterson, M. L., Repka, M., Zhan, X., 2020. Development of a quantitative method to evaluate the printability of filaments for fused deposition modeling 3D printing. Int. J. Pharm. 588, 119760. https://doi.org/https://doi.org/10.1016/j.ijpharm.2020.119760

Yang, Y., Wang, H., Li, H., Ou, Z., Yang, G., 2018. 3D printed tablets with internal scaffold structure using ethyl cellulose to achieve sustained ibuprofen release. Eur. J. Pharm. Sci. 115, 11-18. https://doi.org/https://doi.org/10.1016/j.ejps.2018.01.005

Zhang, J., Feng, X., Patil, H., Tiwari, R. V, Repka, M. A., 2017. Coupling 3D printing with hot-melt extrusion to produce controlled-release tablets. Int. J. Pharm. 519, 186-197 https://doi.org/https://doi.org/10.1016/j.jjpharm.2016.12.049

Zhu, X., Li, H., Huang, L., Zhang, M., Fan, W., Cui, L., 2020. 3D printing promotes the development of drugs. Biomed. Pharmacother. 131, 110644. https://doi.org/https://doi.org/10.1016/j.biopba.2020.110644

1. A method for preparing 3-D printed tablets, comprising the steps of:
Providing a micro-extrusion printhead having a heatable reservoir, a plunger, a printing nozzle and optionally a heater;
Loading a blend of one or more active pharmaceutical agents (APIs) and a polymer suitable for preparing tablets into said reservoir;
Applying heat such that said blend of polymer and API reaches a temperature that is suitable for extrusion through said nozzle;
Applying pressure on said plunger to result in extrusion of said heated blend of polymer and API.

2. The method of claim 1, wherein the API-polymer powder blend or granules are partially molten or soften in the thermoplastic printhead

3. The method of claim 1, wherein the API-polymer powder blend or granules are printed at 30-60° C. above the glass transition or the melting point of the polymer

4. The method of claim 1, wherein the polymer carrier further comprises of non-ionic or ionic/pH dependent polymer, superdisintegrant, sweetener, flavour or combinations thereof. k

5. The method of claim 1, wherein the polymer carrier vries from 20-90% and more preferably from 40-60%

6. The method of claim 1, wherein the superdisintegrant comprises from 2-10%

7. The method of claim 1, wherein the sweetener or combinations comprise from 0.5-20%

8. The method of claim 1, wherein the flavour comprises from 1-5%

9. The method of claim 1, wherein the ratio of the flavour to sweetener varies from 0.5 to 20%

10. The method according to claim 1, wherein said blend of polymer and API is heated to a temperature of about 50° C. to about 200° C.

11. The method according to claim 1, wherein said API is a BCS Class 1 drug thereof.

12. The method according to claim 1, wherein said API is a BCS Class 2 drug thereof.

13. The method according to claim 1, wherein said API is a BCS Class 3 drug thereof.

14. The method according to claim 1, wherein said API is a BCS Class 4 drug thereof.

15. The method of claim 1, wherein the API comprises the 10-70% of the API-polymer powder blend or granule and most preferably the 30-60%

16. The method of claim 1, wherein the API-polymer powder blend or granules have a melt viscosity of 10-1500 Pa·s at the exit of the nozzle

17. The method of claim 1, wherein the API-polymer powder blend or granules have suitable flowability index, preferably 4-10 and compactibility index 5-20

18. The method of claim 1, wherein the API-polymer powder blend or granules have particle size diameter (D50) varying from 50-1000 μm most preferably from 100-300 μm

19. The method of claim 1, wherein the API and the polymer form a glass suspension (type IV or V) or a glass solution when the solid dosage form is printed

20. The method according to claim 1, wherein the active pharmaceutical ingredient comprises one or more of: astemizole, azelastine, azatadine, brompheniramine, carbinoxamine, cetirizine, chlorpheniramine, clemastine, cyproheptadine, desloratadine, dexbrompheniramine, dexchlorpheniramine, diphenhydramine, fexofenadine, hydroxyzine, levocetirizine, loratadine, phenindamine, pheniramine, phenyltoloxamine, promethazine, pyrilamine, terfenadine, tripelennamine, triprolidine, acetyl dihydrocodeine, benproperine, benzonatate, benzylmorphine, bibenzonium bromide, butamirate, butorphanol, carbetapentane, chlophedianol, clobutinol, clofedanol, cloperastine, codeine, dextromethorphan, dextromethorphan hydrobromide, diacetylmorphine, dibunate, dihydrocodeine, dimemorfan, dimethoxanate, diphenhydramine, dropropizine, droxypropine, ethylmorphine, fedrilate, glaucine, hydrocodone, hydromorphone, isoaminile, laudanum, levodropropizine, levomethadone, levopropoxyphene, meprotixol, methadone, morclofone, nepinalone, nicocodine, nicodicodine, normethadone, noscapine, oxeladin, oxolamine, pentoxyverine, pholcodine, pipazetate, piperidione, prenoxdiazine, tipepidine, zipeprol, acetylcysteine, althea root, ambroxol, antimony pentasulfide, bromhexine, carbocisteine, cincolo, combinations, combinations, creosote, dembrexine hydrochloride, domiodol, dornase alfa, eprazinone, erdosteine, guaiacolsulfonate, guaifenesin, hederae helicis folium, ipecacuanha, letosteine, levo verbenone, mannitol, mesna, neltenexine, potassium iodide, senega, sobrerol, stepronin, tiopronin, tyloxapol, pseudoephedrine, cetirizine, loratadine, fexofenadine, diphenhydramine, levocetirizine, desloratadine, phenol, ethanol, thymol, eucalyptol, ethanol, methyl salicylate, chlorhexidine gluconate, cetylpyridinium chloride, hexetidine, triclosan, hydrogen peroxide, domiphen bromide, bismuth subsalicylate, loperamide hydrochloride, cimetidine, famotidine, nizatidine, ranitidine, lansoprazole, omeprazole, esomeprazole, rabeprazole, pantoprazole, dexlansoprazole, diphenoxylate, dicyclomine, loperamide, rifaximin, alosetron, cholestyramine, linaclotide, lubiprostone, polycarbophil, psyllium, alclometasone, amcinonide, beclometasone, betamethasone, budesonide, ciclesonide, clobetasol, clobetasone, clocortolone, cloprednol, cortivazol, deflazacort, deoxycorticosterone, desonide desoximetasone, dexamethasone, diflorasone, diflucortolone, difluprednate, fluclorolone, fludrocortisone, fludroxycortide, flumetasone, flunisolide, fluocinolone acetonide, fluocinonide, fluocortin, fluocortolone, fluorometholone, fluperolone, fluticasone, fluticasone propionate, fluprednidene, formocortal, halcinonide, halometasone, hydrocortisone aceponate, hydrocortisone buteprate, hydrocortisone butyrate, loteprednol, medrysone, meprednisone, methylprednisolone, methylprednisolone aceponate, mometasone furoate, paramethasone, prednicarbate, prednisone, prednisolone, prednylidene, rimexolone, tixocortol, triamcinolone, ulobetasol, 5-fluorouracil, 5-fluorodeoxyuridine, capecitabine, calcium supplements, calcimimetics, cinacalcet, nicotine, nicotine polacrilex, bupropion, varenicline, disulfiram, calcium carbimide, acamprosate, naltrexone, buprenorphine, methadone, levacetylmethadol, lofexidine, betahistine, cinnarizine, flunarizine, acetylleucine, gangliosides, ganglioside derivatives, tirilazad, riluzole, xaliproden, hydroxybutyric acid, amifampridine, doxylamine, diphenhydramine hydrochloride, melatonin, 1-theanine, monofluorophosphate, lactoferrin, lysozyme, lactoperoxidase, glucose oxidase, mutanase, benzocaine, lidocaine, clove oil, sodium bicarbonate, citric acid, tartaric acid, aspirin, ibuprofen, aceclofenac, acemetacin, aloxiprin, azapropazone, benorilate, bromfenac, carprofen, celecoxib, choline magnesium salicylate, diclofenac, diflunisal, etodolac, etoricoxib, faislamine, fenbufen, fenoprofen, flurbiprofen, indometacin, ketoprofen, ketorolac, lomoxicam, loxoprofen, meloxicam, meclofenamic acid, mefenamic acid, meloxicam, metamizole, methyl salicylate, magnesium salicylate, nabumetone, naproxen, nimesulide, paracetamol, oxyphenbutazone, parecoxib, phenylbutazone, piroxicam, salicyl salicylate, sulindac, sulfinpyrazone, suprofen, tenoxicam, tiaprofenic acid, tolmetin, valdecoxib, acetylsalicylic acid, aloxiprin, aminophenazone, anilides, benorilate, benzomorphan derivatives, bezitramide, bucetin, buprenorphine, butorphanol, carbasalate calcium, choline salicylate, codeine, dextromoramide, dextropropoxyphene, dezocine, diamorphine, diflunisal, dihydrocodeine, dihydrocodone, dihydromorphine, diphenylpropylamine derivatives, dipyrocetyl, ethenzamide, fentanyl, floctafenine, flupirtine, glafenine, guacetisal, hydrocodone, hydrocodone bitartrate, hydromorphone, hydromorphone hydrochloride, imidazole salicylate, ketobemidone, metamizole sodium, methadone, morphinan derivatives, morphine, morphine sulphate pentahydrate, morphine-6-glucuronode, morpholine salicylate, nalbuphine, natural opium alkaloids, nefopam, nicomorphine, nifenazone, non-steroidal anti-inflammatory drugs (NSAID), norhydrocodone, noroxycodone, opioids, opium, oripavine derivatives, oxycodeine, oxycodone, oxycodone hydrochloride, oxymorphone, papaveretum, pentazocine, pethidine, phenacetin, phenazocine, phenazone, phenylpiperidine derivatives, piritramide, potassium salicylate, propacetamol, propyphenazone, pyrazolones, rimazolium, salicylamide, salicylic acid derivatives, salsalate, sodium salicylate, tapentadol, tilidine, tramadol, viminol, ziconotide, vitamin B12, cyanocobalamin, riboflavin, guarana, L-carnitine, vitamin A (retinol), B1 (thiamine), B2 (riboflavin), B complex, B6 (pyridoxine), B12 (cobalamin), C (ascorbic acid), D (cholecalciferol), E (tocopherol), F (linoleic acid), G, H (biotin), and K, and choline, folic acid, inositol, niacin, pantothenic acid, para-aminobenzoic acid, terpenoids (e.g., carotenoid terpenoids and non-carotenoid terpenoids), resveratrol, phytosterols, anthraquinones, capsaicin, chlorophyll, betaine, oxalic acid, acetyl-L-carnitine, allantoin, androstenediol, androstendione, betaine (trimethylglycine), calcium pyruvate (pyruvic acid), camitine, carnosine, carotene, carotenoid, choline, chlorogenic acid, cholic acid, chondroitin sulfate, chondroitin sulfate, cholestan, chrysin, coenzyme Q10, conjugated linoleic acid, corosolic acid, creatine, dehydroepiandrosterone, dichlorophen, diindolymethane, dimethylglycine, dimercapto succinic acid, ebselen, ellagic acid, enzymes, fisetin, formononetin, glucaric acid (glucarate), glucosamine (HCl or sulfate), glucosamine (N-acetyl), glutathione, hesperidine, hydroxy-3-methylbutyric acid, 5-hydroxytryptophan, indole-3-carbinol, inositol, isothiocyanates, linolenic acid-gamma, lipoic acid (alpha), melatonin, methylsulfonylmethane, naringin, pancreatin, para-aminobenzoic acid, paraben (methyl or propyl), phenolics, phosphatidylcholine, phosphatidylserine, phytosterols, progesterone, pregnenolone, omega-3 fatty acids, quercetin, resveratrol, D-ribose, rutin, S-adenosylmethionine, salicylic acid, sulforaphane, tartaric acid, taxifolin, tetrahydropalmatine, theophyline, theobromine, tigogenin, troxerutin, or mixtures or combinations thereof.

21. The method according to any of the above claims wherein said tablet is chewable and taste-masked.

22. The method according to any of the above claims wherein said tablet is a pediatric, chewable, taste-masked formulation.

23. The method according to any of the above claims wherein said polymer is selected from ethyl cellulose, carboxymethyl cellulose, hydroxyethyl and hydroxypropyl celluloses, hydroxypropyl methyl cellulose, cellulose acetate phthalate, polyurethane, silicones polycarbonate, polychloroprene, polyisobutylene, polycyanoacrylate, poly (vinyl acetate), polystyrene, polypropylene, poly (vinyl chloride), polyethylene, poly (methyl methacrylate), poly (hydroxyethyl methacrylate), acrylic acid and butyl acrylate copolymer, 2-ethylhexyl acrylate and butyl acrylate copolymer, vinyl acetate and metbyl acrylate copolymer, ethylene vinyl acetate and polyethylene terephthalate, ethylene vinyl acetate and polyethylene, polyethylene and polyethylene terephthalate, or mixtures or combinations thereof.

24. The method according to any of claims 1-22 wherein said polymer is selected from poly (acrylic acid), poly (ethylene oxide) poly (ethylene glycol), poly(vinyl pyrrolidone), poly(vinyl alcohol) polyacrylamide, poly (isopropyl acrylamide) and poly (cyclopropyl methacrylamide), starch, sodium starch glycolate, (lactide-co-glycolide) polymers, aliginic acid, carrageenan chitosan, hyaluronic acid and pectinic acid, or mixtures or combinations thereof.

25. The method according to any of claims 1-22 wherein said polymer is selected from soluplus, PVP/VA 64 and Eudragit EPO.
